(19) United States
(12) Patent Application Publication
(10) Pub. No.: US 2019/0127450 A1
(43) Pub. Date: May 2, 2019

(54) Therapy of multiple organ system failure by anti-toxin antibodies and prophylaxis by anaerobic toxoids

(71) Applicants: Medhat Khafagy, Cairo (EG); Ayatallah Khafagy, Seattle, WA (US)

(72) Inventors: Medhat Khafagy, Cairo (EG); Ayatallah Khafagy, Seattle, WA (US)

(21) Appl. No.: 15/796,839

(22) Filed: Oct. 29, 2017

(51) Int. Cl. C07K 16/12; A61K 39/02

(52) U.S. Cl. CPC C07K 16/1282; A61K 39/02; A61K 2039/55; C07K 2317/70; C07K 2317/92

(57) ABSTRACT

Multiple organ system failure constitutes the most common cause of mortality in intensive care units all over the world. About more than 70 thousand deaths from multiple organ system failure occur every year in the US only. It has been postulated with no definite proof that it is due to translocation of endotoxin from the lumen of the small bowel. Antibodies against anaerobic organisms (mainly clostridia) were found in high concentration in 7 of 8 patients diagnosed with multiple organ system failure from intensive care units in Cairo, Egypt. We suggest that these patients could be saved by antibodies against these toxins prepared in horses injected with anaerobic toxoids, similar to saving patients with toxicity from botulism caused by clostridium botulinum. Prophylaxis against anaerobic sepsis could be achieved by anaerobic toxoids in rats and cohort of 445 patients that underwent major surgery in Cairo, Egypt.

At first, 5 cc specimen of blood will be taken from the patient who was diagnosed with a multiple organ system failure usually residing in an intensive care unit and will be examined for the level of clostridium antitoxin antibodies. If it proved positive we will give these patients antibodies prepared beforehand in horses injected with clostridium toxoid at least 2 weeks in advance. The antibodies will be purified to contain immunoglobulins, humanized and will be tested for allergic reaction in patients before being injected intravenously. The functions of the organs affected by the disease will be tested and monitored by kidney functions (serum creatinine), liver functions by serum albumin and prothrombin time, heart function by ejection fraction on echocardiogram, pulmonary function by FEV1 & 2 and brain function by the consciousness level. We expect about 80% will be rescued by this method similar to that of botulism.

Due to the evolution of multiple drug resistant bacteria, we have to look to ways to increase immunity of patients against it. One of these methods is to raise immunity of patients against anaerobic organisms mainly clostridia by vaccination with anaerobic toxoids. It was tested in a cohort of 445 patients over 6 years by vaccination with anaerobic toxoids about 2 weeks before major surgery. About 50% reduction of surgical site infection was achieved in comparison to the historical control.

Experimental results in rats:
Sepsis after caecal ligation and puncture in Fisher rats (control group)

| | Number of | |
|---|---|---|
| Isolated Organisms | Rats (n = 40) | Percent |
| Escherichia coli | 3 | 7.5% |
| E. coli and C. perfringens | 19 | 47.5% |
| E. coli, C. perfringens | 12 | 30% |
| and Enterococci | | |
| E. coli, and Enterococci | 1 | 2.5% |
| No organisms | 1 | 2.5% |
| Total of dead rats | 36 | 90% |

Sepsis after caecal ligation and puncture in Fisher rats (vaccinated group)

| | Number of | |
|---|---|---|
| Isolated Organisms | Rats (n = 40) | Percent |
| Escherichia coli | 16 | 40% |
| E. coli and C. perfringens | 0 | 0% |
| E. coli, C. perfringens | 0 | 0% |
| and Enterococci | | |
| E. coli, and Enterococci | 0 | 0% |
| No organisms | 0 | 0% |
| Total of dead rats | 16 | 40% |

Survival after caecal ligation and puncture in vaccinated and control

| | Number of rats | | |
|---|---|---|---|
| | survived | Total Number of rats | Survival Percent |
| Control | 4 | 40 | 10% |
| Vaccinated | 24 | 40 | 60% |

Results:
After a successful experiment in combating peritonitis in the model of caecal ligation and puncture by an anaerobic vaccine covexin10 in rats and successful reports in veterinary medicine of preventing sudden death from toxemia due to an anaerobic infection in the rumen of cattle and sheep by the same vaccine, it was decided to undergo a clinical study.
   Range: 15-85 years
   Median: 55 years
   Gender: male & female
   Period: 2010-2015
445 patients who underwent major and semi-major operations were vaccinated each with 0.25 mL of covexin 10 before operation.
Infection of the wounds occurred in 24 of 445 patients (5.4%).
Infection occurred in 7 of 202 who underwent breast operations, mostly for breast cancer including axillary dissection (3.5%).
Infection occurred in 2 of 50 who underwent major operation for colorectal cancer (4%).
3 of 43 patients who underwent major operation for urinary bladder cancer developed wound infection (7.0%).
Other operations for other sites were associated with a low incidence of wound infection compared to historical control.
Although, it was a large series of major operations, not a single case developed multiple organ system failure.
Incidence of infections as regards diabetes

| | | No. of infections | Percentage |
|---|---|---|---|
| No. of | 306 | 14 | 4.6 |
| nondiabetic | | | |
| patients | | | |
| No. of diabetic | 139 | 10 | 7.2 |
| patients | | | |
| Total | 445 | 24 | |

General incidence of infection in the whole series

| No. of infections | No. of patients | Percent |
|---|---|---|
| 24 | 445 | 5.4 |

Incidence of infection according to type of operation

| | | No. of | |
|---|---|---|---|
| Operation | No. of infections | patients | Incidence |
| Breast operation | 7 | 202 | 3.5 |
| Urinary bladder operations | 3 | 43 | 7.0 |
| Gynecological operations | 4 | 23 | 17.4 |
| Colorectal operations | 2 | 50 | 4.0 |
| Head and neck operations | 2 | 32 | 6.2 |
| Operation for malignant | 1 | 4 | 25.0 |
| melanoma | | | |
| Kidney operations | 2 | 8 | 25.0 |
| Operations of skin tumors | 1 | 3 | 33.3 |
| Biliary operations | 1 | 18 | 5.5 |
| Adrenal gland operations | 1 | 2 | 50.0 |
| Soft tissue operations | 0 | 26 | 0 |
| Radical prostatectomy | 0 | 3 | 0 |
| Liver operations | 0 | 12 | 0 |
| Small bowel operations | 0 | 2 | 0 |
| Lung operations | 0 | 4 | 0 |
| Gastric operations | 0 | 9 | 0 |
| Miscellaneous operations | 0 | 4 | 0 |
| | 24 | 445 | |

1-4. (canceled)

5. A method of preventing multiple organ system failure, comprising administering an effective amount of toxoid Merck Sharp and Dome anaerobic toxin Co vaccine 10 to a human patient.

6. The method of claim 5, wherein multiple organ system failure is in intensive care units.

7. The method of claim 5, wherein multiple organ system failure is in the post-operative period.
